(19) United States
(12) Patent Application Publication
(10) Pub. No.: US 2008/0014617 A1
(43) Pub. Date: Jan. 17, 2008

(54) PROCESS AND PLANT FOR THE PRODUCTION OF FERMENTABLE SUGARS FROM CELLULOSE MATERIAL

(75) Inventor: Giuseppina Cerea, Rozzano (Milano) (IT)

Correspondence Address: LUCAS & MERCANTI, LLP, 475 PARK AVENUE SOUTH, 15TH FLOOR, NEW YORK, NY, 10016, US

(73) Assignee: VOMM IMPIANTI E PROCESSI S.P.A., Rozzano (Milano) (IT)

(21) Appl. No.: 11/736,997

(22) Filed: Apr. 18, 2007

(30) Foreign Application Priority Data
Apr. 21, 2006 (EP) 06425275.2

(51) Int. Cl. C12P 19/02; B01J 8/00

(52) U.S. Cl. 435/105; 422/187

(57) ABSTRACT

A process for the production of simple sugars starting from a cellulose material, comprising the steps of:
feeding a continuous flow of the cellulose material or a mixture composed of the cellulose material and water to a turbo-reactor (A) comprising a horizontal axis cylindrical tubular body (1), provided with at least one opening (5, 6) for introducing the cellulose material or the mixture and reagents, at least one discharge opening (7) of the treated material, a heating jacket (4) for bringing the inner wall of the tubular body (1) to a predetermined temperature, a bladed rotor (8), rotatably supported in the cylindrical tubular body where it is put in rotation at speeds in the range of 15-40 m/s, so as to disperse said continuous flow of cellulose material or its mixture in a flow of cellulose material particles, feeding into said turbo-reactor (A), in counter-current with the flow of cellulose material or with the mixture, a continuous and finely subdivided flow of a reagent capable of breaking the integrity of the molecular structure of the cellulose material, making it particularly accessible for being digested during an enzymatic treatment,
centrifuging the particles of cellulose material and the reagent against the inner wall of the turbo-reactor, heated to a temperature of at least 150° C., with formation of a highly turbulent, dynamic, thin tubular fluid layer, wherein the cellulose material particles and the reagent are mechanically maintained in close contact by the blades (9) of the bladed rotor (8),
making the cellulose material and the reagent react in the thin layer while it advances in substantial contact with the inner wall of the turbo-reactor (A) towards the discharge opening (7), continuously discharging a flow of treated cellulose material, sending the flow of treated cellulose material to an enzymatic digestion step with the addition of a sufficient quantity of specific enzymes for hydrolyzing the treated cellulose material, with the obtainment of said simple sugars.

### FIELD OF APPLICATION

In its most general aspect, the present invention refers to the field of the treatment of cellulose material and in particular the invention regards a process and a plant for the production of fermentable sugars, here identified as simple sugars, and/or bioethanol from such material.

By cellulose material it is here intended that material comprising the lignocellulose portion of the biomass that is the fibrous portion of vegetable origin of the biomass and comprising wood, cut branches and rejects from the wood industry in general, forage, vegetables generally including their fruits, seeds and shells comprising the cereals, agricultural and zootechnic residues, rejects from agrobusiness, municipal solid waste, but also paper material, generally as rejects of the paper industry and the like.

### PRIOR ART

The need to have available energy sources which are alternative to traditional sources is known, as is in particular the need to have available biofuels as substitutes of the fossil fuels.

The advantageous characteristics of such biofuels are well known, such as for example bioethanol and biogas, which can be produced from widely available and economical basic material, even by recycling waste materials, and which moreover lead to reduced environmental impact both relative to the production and above all for that concerning their use, for example, as fuel employed in the transportation sector.

To satisfy the aforesaid need, the prior art makes available numerous processes which use the biomass as starting material, which as is known comprises substances of biological origin including materials and residues of agricultural and forest origin, secondary products and rejects of agrobusiness, refluent of zootechnic origin, but also urban waste, the algae and various vegetable species employed in the treatment of organic sewage.

Production processes are known for example of bioethanol obtained through the alcoholic fermentation of simple sugars, such as mainly hexose and pentose monosaccharides, in turn obtained through enzymatic digestion (saccharification) of the fibrous portion present in the biomass, which is appropriately treated.

Examples of such processes are described in the patents U.S. Pat. No. 5,837,506 and EP 0 884 391.

### SUMMARY OF THE INVENTION

One object of the present invention is that of making available an alternative process to the processes made available by the prior art for the production of simple sugars and/or bioethanol starting from a cellulose material, in particular a process which achieves a high yield of conversion of said cellulose material into fermentable sugars and/or bioethanol, that is a process through which the entire lignocellulose portion present in said cellulose material is utilized for the production of such sugars and/or bioethanol.

The aforesaid lignocellulose portion essentially consists of the fibrous portion of vegetable origin of the biomass and mainly comprises lignin, cellulose and hemicellulose.

A further object of the present invention is to make available a process of the aforesaid type which is particularly simple and economically advantageous.

Such objects are achieved, according to the invention, from a process for the production of simple sugars comprising the steps of:
a) feeding a continuous flow of the aforesaid cellulose material or a mixture composed of said cellulose material and water to a turbo-reactor comprising a horizontal axis cylindrical tubular body, provided with at least one opening for introducing the cellulose material or aqueous mixture of cellulose material and reagents, at least one discharge opening of the treated material, a heating jacket for bringing the inner wall of the tubular body to a predetermined temperature, a bladed rotor, rotatably supported in the cylindrical tubular body, where it is put in rotation at a speed in the range of 15-40 m/s, so as to disperse said continuous flow of cellulose material or said mixture in a flow of cellulose material particles,
b) feeding into said turbo-reactor, in counter-current with said flow of cellulose material or with said mixture, a continuous and finely subdivided flow of a reagent capable of breaking the integrity of the molecular structure of said cellulose material, making it particularly accessible for being digested during an enzymatic treatment
c) centrifuging said particles of cellulose material and said reagent against the inner wall of the turbo-reactor, heated to a temperature of at least 150° C., with the formation of a highly turbulent, dynamic and thin tubular fluid layer, wherein the cellulose material particles and said reagent are mechanically maintained in close contact by the blades of said bladed rotor,
d) making said cellulose material and said reagent react in said thin layer while it advances in substantial contact with said inner wall of the turbo-reactor towards the discharge opening,
e) continuously discharging a flow of treated cellulose material, and
f) sending said flow of treated cellulose material to an enzymatic digestion step (saccharification) with the addition of a sufficient quantity of enzymes, said enzymes being specific for hydrolyzing said treated cellulose material with the obtainment of said simple sugars, mainly fermentable monomeric sugars such as hexoses and pentoses, but also disaccharides.

The process according to the invention may optionally comprise a premixing step wherein said continuous flow of cellulose material or said aqueous mixture of cellulose material and said reagent flow are premixed with each other in a turbo-mixer placed upstream of said turbo-reactor.

Such turbo-mixer advantageously comprises a horizontal-axis cylindrical tubular body, provided with inlet openings of said cellulose material or said mixture and said reagent, at least one discharge opening of the cellulose material premixed with said reagent, a cooling/heating jacket for bringing the inner wall of the tubular body to a predetermined temperature in the range of 10-100° C., and a bladed rotor, rotatably supported in the cylindrical tubular body where it is put in rotation at a peripheral speed in the range of 15-40 m/s, so as to premix the continuous flow of said cellulose material or said aqueous mixture of cellulose material with said reagent flow in a flow of premixed material.

The premixed material flow discharged from said turbomixer is then stored in a hopper connected to at least one feeding pump for feeding said premixed cellulose material flow into said turbo-reactor at a pressure in the range of approximately 1-15 bars.

In the case of premixing in said turbomixer, the aforesaid reagent capable of breaking the integrity of the molecular of the cellulose material is preferably sulphuric acid or a solution of sulphuric acid fed in a percentage in the range of 1-5% in relation to the weight of the cellulose material.

It should be added that in the aforesaid case, the process according to the invention may also comprise a neutralization step, for example, by means of adding lime, to block the breaking reaction of the molecular integrity of the lignocellulose portion of the cellulose material, preventing the formation of undesired reaction products such as furfurals.

The furfurals in fact inhibit the enzymatic hydrolysis of the complex sugars and also the alcoholic fermentation of the simple sugars which lead to the formation of bioethanol.

Through such neutralization step, which may advantageously be conducted in a conventional manner before the aforesaid enzymatic digestion step, the pH of the treated cellulose material flow is brought to a value in the range of 1-6.

Advantageously, according to the invention, said reagent capable of breaking the integrity of the molecular structure of the cellulose material may also be an hydrogen peroxide solution.

In the latter case, the hydrogen peroxide solution is advantageously fed directly into said turbo-reactor in counter-current with said cellulose material flow or said aqueous mixture of cellulose material.

The hydrogen peroxide solution may be introduced in the turbo-reactor right at the inlet, since in such case the bladed rotor causes an effective nebulization and centrifugation, consequently ensuring its insertion in the dynamic, turbulent, thin layer of cellulose material particles to be treated in a dispersed condition.

In such a manner, the closest contact possible between the particles and the oxygenated water is favored, and this permits greatly increasing the effectiveness of the treatment.

Alternatively, or in addition to the aforesaid insertion through an inlet opening, the introduction of hydrogen peroxide can also be carried out through openings for the nebulization of hydrogen peroxide placed at different levels along the length of the cylindrical tubular body.

That said above regarding the hydrogen peroxide is valid for any employed reagent capable of breaking the integrity of the molecular structure of the cellulose material, even if in the case of sulphuric acid and similar reagents a process comprising the aforesaid premixing step is preferred, as described above.

The hydrogen peroxide solution is fed at a pressure in the range of about 1 bar to about 15 bars and is composed of hydrogen peroxide to at least 10 volumes (i.e. 3% titer), preferably to 30-40 volumes.

The weight ratio between hydrogen peroxide employed and cellulose material varies from 1:2 to 1:20.

The residence time of said flow of cellulose material or said mixture of water and cellulose material, with said reagent flow in the turbo-reactor, is a variable time depending on the type and quantity of employed reagent, as well as on the temperature of the inner wall of the turbo-reactor.

The reaction in the turbo-reactor has in fact the object of making the cellulose material particularly accessible, particularly its fibrous or lignocellulose portion, for the subsequent step of enzymatic hydrolysis, mainly through the breaking of the crystalline structure of the lignin present in the cellulose material.

For example, employing a solution of 5% hydrogen peroxide (16.6 volumes), fed into the turbo-reactor at double the flow rate of the cellulose material flow, the aforesaid residence time is preferably in the range of about 3 minutes to about 30 minutes, and more preferably is a residence time of about 10 minutes.

Advantageously, then, the turbo-reactor is a substantially sealed turbo-reactor which is capable of maintaining an inner pressure at a value of about 15 bars, or rather a pressure essentially corresponding to that of the flow or flows fed into it.

Regarding the aforesaid step of enzymatic digestion or saccharification, it should be said that it is carried out at a temperature in the range of 35-45° C., preferably 40° C., and in any case corresponding with a temperature at which the activity of the employed enzymes is sufficient to achieve the aforesaid hydrolysis reaction, through which the complex sugars (polysaccharides and/or oligosaccharides) are transformed into simple fermentable sugars.

For such purpose, the flow of treated cellulose material discharged from the turbo-reactor may be subjected to cooling, also for example by dilution.

In any case it should be said that the temperature of the treated cellulose material flow at the turbo-reactor's discharging mainly depends on the temperature of the inner wall of the turbo-reactor and on the residence time of the same cellulose material flow at its interior.

Moreover, it should be said that the aforesaid step of enzymatic digestion is carried out for a time which varies in the range of 10 minutes to 36 hours, depending on the enzymes employed, that is, on their activity and the quantity used.

In particular, any enzyme of known type can be employed which is capable of inducing the aforesaid step of enzymatic digestion or saccharification, and only as an example we mentioned the enzymes available with the trademark of Celluclast and Liquozyme® SC.

The enzymatic digestion step can moreover be conducted through a plurality of substeps of enzymatic digestion, in each of which predetermined quantities of enzymes may be added, even of different type.

Regarding once again the cellulose material, it should be said that it is fed in a flow with a rather fine particle size, wherein preferably no more than 10% of the cellulose material is of size greater than 1000 microns and in any case less than 1500 microns.

Still more preferably, at least 50% of the cellulose material has a particle size comprised between 200 and 1000 microns.

It should be said that the process according to the invention may involve a dilution, generally with water, of the flow of treated cellulose material discharged from the turbo-reactor, or said flow discharged from the turbo-reactor can be if necessary dried.

This is verified when, for a flow of treated cellulose material discharged from the turbo-reactor, a mass of treated cellulose material is desired with specific fluidity and/or moisture characteristics.

On the other hand, as it appears from that stated above, such characteristics can be attained by causing an appropriate mixing of the cellulose material fed into the turbo-reactor together with water.

Regarding the temperature of the inner wall of the turbo-reactor, which as previously set forth is heated to at least 150° C., it should be said that it is preferably in the range of about 220 to about 300° C., best if about 260° C.

The process according to the invention may also comprise, subsequently or simultaneously with the aforesaid enzymatic digestion step, a step of alcoholic fermentation, of known type, with the transformation of said simple sugars into bioethanol.

In another of its aspects, the present invention refers to a plant for carrying out the above illustrated process which comprises a turbo-reactor of the type described above and optionally a turbo-mixer, it too of the above described type placed upstream of the turbo-reactor.

Such plant may moreover comprise a hopper placed downstream of the turbo-mixer and a pump connected with said hopper and placed upstream of said turbo-reactor, respectively for the collection of the aforesaid flow of premixed cellulose material discharged from the turbo-mixer and for feeding the flow collected in said hopper into the turbo-reactor at a pressure in the range of about 1 bar to about 15 bars.

It should be said that, advantageously, the present process is particularly effective, giving high conversion yields.

In particular, it should be noted that such yields are surprising high in the case a solution of hydrogen peroxide is employed as reagent inside the turbo-reactor.

The use of hydrogen peroxide in the turbo-reactor moreover has the advantage of not influencing the activity of the enzymes employed in the subsequent step of enzymatic digestion, to which the treated cellulose material discharged from the turbo-reactor is subjected.

The hydrogen peroxide in fact is completely deactivated after the reaction in the turbo-reactor.

Consequently, no neutralization step is requested for the cellulose material before the saccharification step, as instead is in the case of the use of sulphuric acid which must be neutralized with an oxide or alkaline hydroxide or earth alkaline, for example with lime.

Again, the residual cellulose material which is obtained after the step of enzymatic digestion, in case the aforesaid reaction in the turbo-reactor was conducted with hydrogen peroxide, is composed of an active fiber which advantageously may be employed as a dietary supplement in the human diet, for example as a dietary supplement of fibers and salts.

Further advantages and features of this invention will be more evident from the description of an embodiment of a process according to the invention, hereinafter provided with reference to the attached drawing, given for illustrative and non-limiting purposes.

### BRIEF DESCRIPTION OF THE DRAWINGS

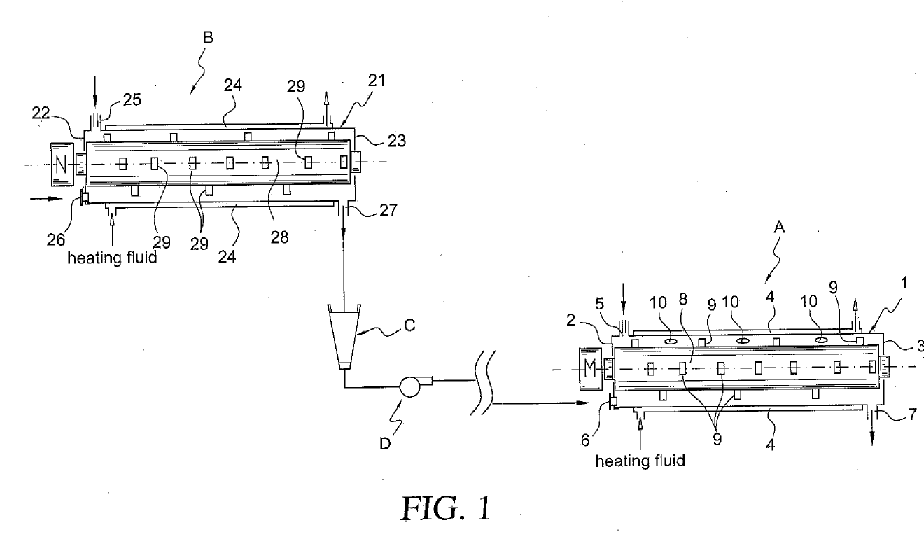

In FIG. 1, several details are schematically represented of a plant for the production of simple sugars and/or bioethanol from a cellulose product according to the process of the invention.

### DETAILED DESCRIPTION OF THE INVENTION

With reference to FIG. 1, the turbo-reactor A is essentially composed of a cylindrical tubular body 1, which is substantially sealed for pressure values up to 15 bars, closed at opposite ends by bottoms 2, 3 and coaxially equipped with a heating jacket 4 intended to be run through by a heating fluid, for example steam under pressure or diathermic oil, to maintain the inner wall of the body 1 at a predetermined temperature.

The tubular body 1 is provided with inlet openings 5, 6 of the cellulose material to be treated or a mixture of it with water and respectively of the reagent employed for breaking the crystalline structure of the fiber present in the cellulose material, as well as a discharge opening 7 of the treated cellulose material.

In the tubular body 1, a bladed rotor 8 is rotatably supported whose blades 9 are helically arranged and are oriented for centrifuging and simultaneously conveying the cellulose material subjected to treatment towards the outlet.

A motor M is provided for the operation of the bladed rotor at peripheral speeds which vary from 15-40 meters per second.

In the inner wall of the cylindrical tubular body 1, openings 10 are provided for the insertion of the aforesaid reagent in nebulized form.

This, in particular, if an hydrogen peroxide solution employed as reagent is utilized.

A plant for producing simple sugars and/or bioethanol according to the process of the invention comprises, in addition to the turbo-reactor A, a turbo-mixer B for premixing the cellulose material or a mixture of water and cellulose material with the employed reagent, in the case the latter is, preferably, sulphuric acid or a solution of this.

In particular, the turbo-mixer B is put upstream of the turbo-reactor A and essentially comprises a cylindrical tubular body 21, closed at the opposing ends by bottoms 22, 23 and coaxially equipped with a heating/cooling jacket 24 intended to be run through by a fluid, for example diathermic oil for maintaining the inner wall of the body 21 at a predetermined temperature.

The tubular body 21 is provided with an inlet opening 25 of the cellulose material or a mixture of water and cellulose material, an inlet opening 26 of the sulphuric acid solution, and a discharge opening 27 of the cellulose material at the end of the premixing step.

For the feeding of solid flows and liquid flows, the aforesaid openings are respectively equipped with a high speed screw feeder and with controlled injection tubes, neither shown in the figures.

In the tubular body 21, a bladed rotor 28 is rotatably supported which is entirely similar to the bladed rotor 8 of the turbo-reactor A, hence it too comprises blades, here indicated with 29, and is rotatably supported in the cylindrical tubular body where it is put in rotation at peripheral speeds in the range of 15-40 m/s, so as to mix the continuous flow of cellulose material or the mixture of water and cellulose material with the sulphuric acid in a premixed flow.

The bladed rotor is driven into rotation by a motor N and it is intended to convey the cellulose material or mixture of water and cellulose material subjected to the premixing with the sulphuric acid towards the outlet 27.

At the outlet 27, the premixed cellulose material is discharged into a collection and storage hopper C which is connected to at least one feed pump D for feeding the flow of premixed cellulose material into the turbo-reactor A at a temperature in the range of about 1 to about 15 bars.

Without wishing to limit the scope of the present invention, the following example illustrates the manner in which the present invention may be carried out and used.

In particular, in the following example, specific reference is made to the bran as residue of the grinding of cereals mainly composed of seed cods, but it must be understood that other cellulose materials, such as those for example mentioned above (but not only these) can be employed in the process according to the present invention.

### EXAMPLE

To a turbo-reactor A made of special Uranus B6 alloy, whose bladed rotor is made to turn at a peripheral speed of about 35 m/s and whose inner wall is maintained at 260° C., a continuous flow of native wheat bran, with flow rate of 20 kg/hour, is fed through the opening 5.

Simultaneously, through the opening 6 and the openings 10, a 5% hydrogen peroxide solution (16.6 volumes) is continuously fed at 40 kg/hour at room temperature.

The bran has a particle size which has the following distribution:
6%>1400 microns
26.8%>1000 microns
61.9%>200 microns
3.6%>100 microns
1.8%<100 microns

At the inlet of the turbo-reactor A, the bran flow is mechanically dispersed into small particles which are immediately centrifuged against the inner wall of the turbo-reactor itself, where they form a dynamic, thin tubular layer.

Simultaneously, the hydrogen peroxide solution entering through the opening 6 is mechanically, finely nebulized by the blades 9 of the rotor 8, which also cause the immediate centrifugation of the smallest obtained drops. These are thus inserted in the dynamic, thin tubular layer of bran particles, with which they can interact.

The hydrogen peroxide solution introduced in nebulized form through the openings 10 further increases the interaction of the hydrogen peroxide with the bran particles.

After a residence time of about 9 minutes in the turbo-reactor A, a flow of treated bran is continuously discharged from the opening 7 with a moisture content of about 61%, with a temperature of about 100° C. and with a pH of 5.6-5.7.

The treated bran presents itself at the discharge as a bleached moist mass with considerably modified fiber.

The capacity of such product to provide simple sugars was then verified by means of enzymatic hydrolysis.

The treated bran exiting from the turbo-reactor A was diluted with water 1.1 to a temperature of about 15° C., obtaining a treated bran mass at about 35° C.

A portion of said treated bran mass, in particular 200 g, is further diluted with 400 g of cold water, to obtain a mass which can be stirred with magnetic armature.

Then, a stirrer and the beaker containing the obtained treated bran solution are put in a stove at 40° C., where they are maintained until the end of the process for obtaining fermentable sugars.

At this point, a first quantity of enzymes is added to the aforesaid solution in the form of 2.4 g of Celluclast® 1.5 L FC tq.

A subsequent first measurement of the sugars in solution carried out after about an hour from the first addition of enzymes reveals a content of sugars equal to about 26 mmol/l, i.e. 4.7 g/l.

The determination of the sugars in solution is carried out with an instrument commercialized as Accu-Check Comfort© by Roche Diagnostics, employed for personal blood glucose control, which has a measurement range of 0.36-4.5 g/l.

A second and a third measurement carried out at subsequent hourly intervals do not reveal appreciable variations of the content of sugars in solution, therefore another 2.4 g of Celluclast® 1.5 L FC tq are added without leading in any case to substantial variations of the sugar content calculated in the space of about an hour from the last addition.

Then 1 g of Liquozyme© SC is added to the treated bran solution.

After about an hour from this addition, 32.7 mmol/l, i.e. 5.8 g/l of sugars in solution were detected.

The solution was maintained in the stove overnight, as said at 40° C., and the following morning the Accu-Check© displays over-range during repeated measurements.

The solution is therefore diluted 1.10 with water and at the subsequent measurement, the Accu-Check© shows a content of sugars of 17.5 mmol/l, i.e. 3.15 g/l.

From this last concentration, it is calculated that starting from 40 g of bran (dry substance), 18.8 grams of sugars are obtained with a yield therefore of 47%.

1. Process for the production of simple sugars starting from a cellulose material, comprising the steps of:
a) feeding a continuous flow of the said cellulose material or a mixture composed of said cellulose material and water to a turbo reactor comprising a horizontal axis cylindrical tubular body, provided with at least one opening for introducing said cellulose material or said mixture and reagents, at least one discharge opening of the treated material, a heating jacket for bringing the inner wall of the tubular body to a predetermined temperature, a bladed rotor, rotatably supported in the cylindrical tubular body where it is put in rotation at speeds in the range of 15-40 m/s, so as to disperse said continuous flow of cellulose material or said mixture in a flow of cellulose material particles,
b) feeding into said turbo-reactor, in counter-current with said flow of cellulose material or with said mixture, a continuous and finely subdivided flow of a reagent capable of breaking the integrity of the molecular structure of said cellulose material, making it particularly accessible for being digested during an enzymatic treatment,
c) centrifuging said particles of cellulose material and said reagent against the inner wall of the turbo-reactor, heated to a temperature of at least 150° C., with formation of a highly turbulent, dynamic, thin tubular fluid layer, wherein the cellulose material particles and said reagent are mechanically maintained in close contact by the blades of said bladed rotor,
d) making said cellulose material and said reagent react in said thin layer while it advances in substantial contact with said inner wall of the turbo-reactor towards the discharge opening,
e) continuously discharging a flow of treated cellulose material, and
f) sending said flow of treated cellulose material to an enzymatic digestion step with the addition of a sufficient quantity of enzymes, said enzymes being specific for hydrolyzing said treated cellulose material with the obtainment of said simple sugars.

2. Process according to claim 1, wherein said flow of cellulose material or said mixture and said reagent flow are premixed with each other in a turbo-mixer placed upstream of said turbo-reactor, said turbo-mixer comprising a horizontal-axis cylindrical tubular body, provided with inlet openings of said cellulose material or said mixture and said reagent, at least one discharge opening of the cellulose material premixed with said reagent, a heating/cooling jacket for bringing the inner wall of the tubular body to a predetermined temperature in the range of 10-100° C., and a bladed rotor, rotatably supported in the cylindrical body where it is put in rotation at peripheral speeds in the range of 15-40 m/s, so as to premix said continuous flow of cellulose material or said mixture with said continuous reagent flow in a continuous flow of premixed material.

3. Process according to claim 2, wherein said continuous flow of premixed material discharged from said turbo-mixer is stored in a hopper connected to at least one feed pump for feeding said continuous flow of premixed cellulose material into said turbo-reactor at a pressure in the range of about 1 to about 15 bars.

4. Process according to claim 1, wherein said reagent capable of breaking the integrity of the molecular structure of said cellulose material is sulphuric acid or a solution of sulphuric acid.

5. Process according to claim 4, wherein said sulphuric acid is fed in a percentage in the range of 15% in relation to the weight of the cellulose material.

6. Process according to claim 1, comprising, before said enzymatic digestion step, a neutralization step in which said treated cellulose material which has been discharged by said turbo-reactor is made to react with an oxide or alkaline hydroxide or earth alkaline.

7. Process according to claim 4, comprising, before said enzymatic digestion step, a neutralization step in which said treated cellulose material which has been discharged by said turbo-reactor is made to react with an oxide or alkaline hydroxide or earth alkaline.

8. Process according to claim 1, wherein said reagent capable of breaking the integrity of the molecular structure of said cellulose material is an hydrogen peroxide solution.

9. Process according to claim 8, wherein said hydrogen peroxide solution is fed at a pressure in the range of about 1 to about 15 bars.

10. Process according to claim 8, wherein said hydrogen peroxide solution consists of hydrogen peroxide of at least 10 volumes (3% titer).

11. Process according to claim 10, wherein said hydrogen peroxide solution consists of hydrogen peroxide of 30-40 volumes.

12. Process according to claim 8, wherein the weight ratio between hydrogen peroxide and cellulose material varies from 1:2 to 1:20.

13. Process according to claim 1, wherein the residence time of said flow of cellulose material or said mixture of water and cellulose material with said reagent flow in said turbo-reactor is a time which varies in the range of about 2 to about 30 minutes.

14. Process according to claim 1, wherein said turbo-reactor is a substantially sealed reactor capable of maintaining an internal pressure up to a value of about 15 bars.

15. Process according to claim 1, wherein said enzymatic digestion step is carried out at a temperature in the range of 35-45° C., preferably at 40° C.

16. Process according to claim 15, wherein said enzymatic digestion step is carried out for a time which varies in the range of 10 minutes to 36 hours.

17. Process according to claim 1, wherein the inner wall of said turbo-reactor is heated to a temperature in the range of about 230 to about 300° C.

18. Process according to claim 1, wherein the inner wall of said turbo-reactor is heated to a temperature of about 260° C.

19. Process according to claim 1, wherein said enzymatic digestion step is followed by or carried out simultaneously with a step of alcoholic fermentation, with consequent transformation of said simple sugars into ethanol.

20. Plant for carrying out the process according to claim 1 comprising:
a) a turbo-reactor comprising a horizontal-axis cylindrical tubular body, provided with at least one opening for introducing said cellulose material or said mixture and reagents, at least one discharge opening of the treated product, a heating jacket for bringing the inner wall of the tubular body to a predetermined temperature, a bladed rotor rotatably supported in the cylindrical tubular body, said turbo-reactor being a substantially sealed reactor capable of maintaining an internal pressure up to a value of about 15 bars, and
b) a turbo-mixer connected upstream to said turbo-reactor and comprising a horizontal-axis cylindrical tubular body, provided with inlet openings, at least one discharge opening, a cooling/heating jacket for bringing the inner wall of the tubular body to a predetermined temperature, and a bladed rotor, rotatably supported in the cylindrical tubular body.

21. Plant according to claim 20, further comprising a hopper placed downstream of said turbo-mixer and a pump connected to said hopper and placed upstream of said turbo-reactor respectively for the collection of said flow of premixed cellulose material discharged from said turbomixer and for feeding said collected flow into said turbo-reactor at a pressure in the range of about 1 to about 15 bars.
